(19) United States
(12) Patent Application Publication
(10) Pub. No.: US 2015/0037885 A1
(43) Pub. Date: Feb. 5, 2015

(54) METHOD OF PRODUCING HUMAN RETINAL PIGMENTED EPITHELIAL CELLS

(71) Applicants: Heli Skottman, Helsinki (FI); Heidi Hongisto, Helsinki (FI); Hanna Hiidenmaa, Helsinki (FI); Jukka Partanen, Helsinki (FI); Kaija Alfthan, Helsinki (FI)

(72) Inventors: Heli Skottman, Helsinki (FI); Heidi Hongisto, Helsinki (FI); Hanna Hiidenmaa, Helsinki (FI); Jukka Partanen, Helsinki (FI); Kaija Alfthan, Helsinki (FI)

(73) Assignee: GLYKOS FINLAND OY, Helsinki (FI)

(21) Appl. No.: 13/996,253

(22) PCT Filed: Dec. 20, 2011
(86) PCT No.: PCT/FI2011/051142

§ 371 Date: Aug. 23, 2013

(30) Foreign Application Priority Data
Dec. 20, 2010 (FI) 20106354

(51) Int. Cl. C12N 5/079

(52) U.S. Cl. CPC C12N 5/0621; C12N 2506/02; C12N 2501/59
USPC 435/368; 435/289.1

(57) ABSTRACT

The production of human retinal pigment epithelial cells having a step of incubating human pluripotent stem cells with binder molecules binding to terminal N-acetyllactosamine (Galβ1-4Glc NAc) and/or blood group H determinant type 2 (Fucα1-2Galβ1-4Glc NAc). A method of using lectin ECA in a culture of human pluripotent stem cells in order to support the stem cells to differentiate into retinal pigment epithelial cells is also disclosed.

The present invention relates to the production of human retinal pigment epithelial cells comprising a step of culturing human pluripotent stem cells with binder molecules binding to terminal N-acetyllactosamine (Galβ1-4GlcNAc) and/or blood group H determinant type 2 (Fucα1-2Galβ1-4GlcNAc). Particularly, the invention relates to a use of lectin ECA in a culture of human pluripotent stem cells in order to support the stem cells to differentiate into retinal pigment epithelial cells. The invention thus provides a novel and effective way to produce retinal pigment epithelial cells for regenerative medicine and for drug screening.

### BACKGROUND OF THE INVENTION

Diseases of eye, such as retinitis pigmentosa, complications of various forms of diabetes or age-related macular degeneration, often lead to blindness due to the loss of photoreceptor cells. In single-gene defects, such as retinitis pigmentosa, gene-therapy may provide one option but for more complex traits, no established treatments are available. One therapeutic option is transplantation of functional photoreceptor cells. Use of functional retina cells of fetal origin has been tested (Radke et al., Arch Ophthalmol 2004; 122: 1159-1165); in addition to obvious ethical questions, this approach may be limited by the availability and quality of the fetal cells. Regeneration of retinal pigment epithelial (RPE) cells is also essential for the therapies, in particular in age-related macular degeneration and its so called wet form, as they, e.g., supply nutrients to the retina and maintain blood-retina barrier. RPE cells and photoreceptors are assumed to form a single unit; degeneration of RPE cells ultimately leads to loss of function of photoreceptors.

One desirable option could be generation of photoreceptors and RPE cells from established human embryonic stem cell (ESC) lines or other pluripotent cell lines such as induced pluripotent stem cells (iPS; see Takahashi et al., Cell 2007; 131: 861-867), as they could provide an unlimited, well-characterized source for the therapeutic cells. Furthermore, there are currently means to establish human ESC lines without destroying embryos, resulting in an ethically more acceptable option.

There, however, still exists a high number of problems related to the quality of ESC lines, their culturing conditions and reliable induction toward retinal direction. There are currently methods to produce RPE cells from human pluripotent stem cells but simple, reliable and effective methods to induce or produce high numbers of these cells are missing (see, e.g., Osakada et al., Nature Biotechology 2008; 26: 215-224; Lamda et al., PNAS 2006; 103: 12769-12774; Viczian et al., PLoS Biology 2009; 7:e1000174; Steedman et al., Biomed Microdevices 2010; 12: 363-369). In certain production models, an induction signal by embryonic retinal tissue to ESC lines has been noted to be essential (see references in Osakada et al., Nature Biotechology 2008; 26: 215-224). In the absence of co-culture with embryonic fetal tissue, differentiation of progenitors into photoreceptors was infrequent, too low for therapeutic production. The nature of the signal or molecules mediating the induction are not currently known. Further, use of animal-derived substances, such as the mouse sarcoma-derived Matrigel as a cell culture support matrix, or fetal bovine serum as an additive in culturing medium lead to expression of unaccepted glyco-structures on cell surface as demonstrated by Martin et al., Nature Medicine 2005; 11: 228-232. Hence, well-defined culturing conditions are needed. As culturing of human ESC lines in general has proved to be difficult (Hoffman and Carpenter, Nature Biotechnology 2005; 23: 699-708), it is even more demanding to culture them in conditions that can produce a high number of desired differentiated cells with therapeutic quality.

In addition to the use of RPE cells in regenerative medicine or therapy, the ability to produce RPE cells, or in fact any other cell type, from their progenitor cells provides an excellent opportunity to screen and develop novel drugs for diseases (Pouton and Haynes, Nat Rev Drug Discov 2007; 8: 605-616). The practically unlimited source of human tissues and cells of various differentiation levels is most valuable for drug development, as hence model systems for primary screening of drugs can be created, secondary screenings related to toxicity and functionality of candidates can be assayed and metabolic profiles become available. Furthermore, the possibility to produce RPE cells, and/or their progenitors and/or tissues derived thereof, by producing iPS cell lines starting from practically any cell from a patient enables even more focused drug screening. The use of patient-derived iPS cell lines also enables studies of pathogenic processes related to the disease in question. For example, it is possible to take patient's skin fibroblasts as starting material and produce pluripotent stem cells from those cells in vitro. It would be highly desirable to be able to efficiently induce various more-differentiated cell lineages or tissues, starting from the patient-derived iPS cells.

iPS cells derived from the non-eye cells of the recipient or from the cells of an individual with HLA genes sufficiently similar to those of the recipient, can be used to avoid immunological rejection of the RPE graft. Similarity of the HLA genes between the recipient and donor is known to decrease the risk for rejection of the allogeneic graft.

Hence, well-characterized, affordable, robust and safe methods for the production of human RPE cells would be highly desirable.

The retina develops from the anterior neural plate, from a region of the developing central nervous system called the diencephalon. Protocols for inducing various types of neurons from human ESC lines have been published. They typically rely on manipulation of developmental signaling pathways. A group of transcription factors that are important, even essential, for development of the eye have been identified (Zuber et al., Development 2003; 130: 5155-5167) and often called the ‘eye field transcription factors’. Measurement of these factors can be applied in methods aiming to induction of e.g. RPE cells from pluripotent stem cells. For example, factors called PAX6, RAX and MITF can be measured using RT-PCR type of methods to estimate the differentiation, or the strength of the induction, toward the retinal lineage. PAX6 is early ectodermal markers, whereas RAX indicates early eye precursor cells, common for neural retina and RPE cells. MITF indicates the RPE cell lineage.

Practically all proteins and some proteins located in or on the surface of human cells are glycosylated, that is, they carry oligosaccharide, ‘glycan’ structures linked to certain amino acids or lipid residues. These glycans mediate various functions, for example, related to targeting of cells onto tissues, regulating their activities or differentiation and intracellular signalling. Due to technology-related limits systematic characterisation of glycan structures (‘glycomes’) of cells or cell surface is just emerging. For example, the N-glycome of human ESC lines was first described by Satomaa et al. (BMC Cell Biol 2009; 10:42). The fine structures of glycans seem to be specific for a cell type and their differentiation level. Further, the glycan structures are species—and cell type specific (Cagneaux and Varki, Glycobiology 1999; 9: 747-755). All this complicates studies on glycans, as no predictions from earlier findings can readily be drawn.

Patent application WO2010 004096 teaches that the lectins, that is, proteins binding to particular glycan structures, specific for and known to bind to terminal N-acetyllactosamine (Galβ1-4GlcNAc) and blood group H determinant type 2 (Fucα1-2Galβ1-4GlcNAc) can be used as culture matrix to cultivate human embryonic stem cells and iPS cells in undifferentiated state in vitro; one such lectin was ECA derived from Erythrina cristagalli . However, the application does not teach anything about the ability to induce RPE cells. In the same line of teaching is Mandai et al. (Cell Transplant 2010; 19: 9-19), who demonstrate that ECA and certain other lectins can be used in negative selection to enrich mouse retinal progenitor cells. It is further known that glycosylation is species specific and cell type specific including differentiated forms (WO 2008 087257).

### DESCRIPTION OF THE DRAWINGS

FIG. 1 shows hESC-derived RPE cells after A) 28 and B) 70 days of differentiation on ECA coated culture plates.

FIG. 2A demonstrates relative quantitative RT-PCR analysis of RPE mRNA markers of human ESCs after 7 days of differentiation on ECA and Matrigel. Expression levels of neuroectodermal (PAX6) and retinal (RAX) precursor genes were increased as compared to undifferentiated hESCs set as the calibrator (equaling to 1).

FIG. 2B demonstrates relative quantitive RT-PCR analysis of RPE mRNA markers of human ESCs after 28 days of differentiation on ECA and Matrigel. Expression levels of

PAX6 and RAX were decreased whereas the expression level of the early RPE marker MITF was increased.

FIG. 3 shows hESC-derived RPE cells after 91 days of differentiation on ECA demonstrating cobblestone morphology of mature RPE cells.

FIG. 4 shows hESC-derived RPE cells after 140 days of differentiation on ECA and immunostained for expression of RPE specific markers MITF, CRALBP, Bestrophin, tight junction protein Zo1, and proliferation marker Ki67. Nuclei were counterstained with DAPI. Scale bars=10 μm.

### DETAILED DESCRIPTION OF THE INVENTION

The present invention is based on the finding that a particular lectin molecule purified from Erythrina cristagalli, usually called ECA, was able to support or strengthen the in vitro differentiation of human pluripotent stem cells toward retina pigment epithelial cells in cell culture conditions. In particular, neuroectodermal (PAX6) and retinal (RAX) precursor genes as well as the early marker for RPE cells, MITF, were upregulated on human embryonic stem cells cultivated in the presence of ECA on day 7. On day 28, the decreasing expression of RAX and PAX6 and enhanced expression of MITF was detected, indicating differentiation toward RPE lineage. The expression of MITF was higher in cells cultured on plates coated with ECA as compared to the controls cultivated on the standard Matrigel matrix. The standard cell culture medium for RPE induction was used (Vaajasaari et al., 2011, J Molecular Vision 17:558-575).

The invention describes a novel set of molecules that efficiently increases the production of high numbers of RPE cells and/or their progenitor stages. There are currently no methods to efficiently increase the induction of human pluripotent stem cells toward the RPE cell lineage using simple, defined molecules.

The RPE cells can be applied in regenerative medicine, to produce therapeutic cells for various eye diseases such as retinitis pigmentosa, complications of diabetes and/or age-related macular degeneration. In a preferred embodiment of the invention, the cells produced are used for cellular therapy for age-related macular degeneration. Protocols for transplantation of RPE cells have been described in the art, for example by Reh and co-workers (Reh et al. in Ding (ed) Cellular Programming and Reprogramming: Methods and Protocols. Methods in Molecular Biology vol 636, pp 139-153). In their mouse model, Reh et al. injected approximately 200 000-300 000 ESC-derived RPE cells in the volume of 2-3 microliter into the subretinal region of eye. The mice were treated with Cyclosporin A prior to operation and anesthetized; the cells were injected using capillaire tips of about 120 micrometer in diameter. To avoid immunological rejection of allogenic (i.e. cells originating from a different individual than the recipient) PRE graft, the RPE cells can be produced starting from iPS cells of the recipient or from iPS cells of an individual whose HLA genes are sufficiently similar to those of the recipient.

The RPE cells and/or their progenitor stages, produced according to the present invention or tissues derived thereof, also can be applied in screening drugs for the eye diseases. The said tissues or cell cultures can further be applied to study effects on eye development of the drug candidates. Furthermore, if induced pluripotent stem (iPS) cells, originating from a patient with an eye disease are induced toward the RPE cell lineage, the invention can be used to study pathogenic processes related to the same disease.

The present invention relates to a method of producing human RPE cells and/or their progenitor stages from pluripotent stem cells in vitro by culturing the pluripotent stem cells in the presence of a binder or binders with the similar binding specificity as lectin ECA, that is, binding to terminal N-acetyllactosamine (Galβ1-4GlcNAc) and blood group H determinant type 2 (Fucα1-2Galβ1-4GlcNAc). The term “terminal” indicates herein a non-reducing end terminal structure, Gal is D-galactopyranose, GlcNAc is D-N-acetylglucosamine in pyranose form, Fuc is L-fucose pyranose form, the glycosidic linkages are indicated as in the art and IUPAC, β1-4 is in condensed form β4, α1-2 is α2. Lectin ECA is purified from Erythrina cristagalli. It has gene databank codes AY158072.1 and GI:37724084, and protein databank code P83410, encoding 239 amino acids and resulting in a protein of 26 231 Da in size. It is obvious to those skilled in the art that proteins with essentially similar features but with some variation in their primary sequences may exist as natural variants. Furthermore, it is obvious that genes and proteins with essentially the same functional properties but with some variation in their primary sequences can be produced using standard recombinant DNA techniques. It is known (Cummings and Etzler, in Varki et al (eds) Essentials of Glycobiology, chapter 45 pp 633-647, 2^{nd} ed, 2009) that also other natural lectins, such as galectin-1, DSA and UEA-1, have a binding specificity essentially similar to that of ECA. An “essentially similar binding specificity” here refers to the ability of a molecule to bind to terminal N-acetyllactosamine (Galβ1-4GlcNAc) and blood group H determinant type 2 (Fucα1-2Galβ1-4GlcNAc) sufficiently to support differentiation of ESC or iPS cells toward RPE. In one embodiment of the invention, galectin-1 and/or DSA and/or UEA-1, or any combinations of them, with or without ECA is used as an additive in the cell culture system aiming to production of RPE cells. It is known in the art that also other molecules than lectins can have similar binding specificities; such molecules can be antibodies or fragments thereof or glycan-binding enzymes, such as a glycosyltransferase, so modified that the binding specificity is intact but the actual enzymatic activity is missing (Aalto et al Glycoconjugate J 18: 751-758, 2001). In an embodiment of the invention, an antibody or modified enzyme with a binding specificity similar to ECA is used for culturing. In the present invention, term ‘binder’ refers to a lectin, antibody, or modified enzyme or similar binding to the said glycan structure. Further, in order to avoid contaminating the cell product, the binder molecule—a lectin, antibody, modified enzyme or equivalent, can be attached or linked, either covalently or non-covalently onto solid phase such as cell culture surface with various techniques known in the art. Examples of the enzymes include N-acetyllactosamine Galβ4GlcNAc modifying enzymes, α3-sialyltransferase, such as α3-sialyltransferase III or IV, α3-fucosyltransferase IV, IX, or V and β4-galactosidase such as Diplococcus pneumoniae β4-galactosidase. In one embodiment of the invention, the cells attached to the binder can be released using competitive sugars or glycans as described, e.g., by Laine et al (WO 2008087257). Examples of antibodies include non-reducing end terminal Galβ4GlcNAc binding antibodies and non-reducing end terminal Fucα2Galβ4GlcNAc binding, H type II, antibodies. The antibody is in a preferred embodiment human monoclonal antibody such as IgG or IgM, or fragment thereof preferably in conjugated form such as glycan such as enzymatically modified or oxidized glycan and/or specific amino acid residue conjugated form, e.g., N-terminal amine, lysine, thiol, carboxylic acid side chains, periodic acid oxidized N-terminal Ser/Thr or Cys (see WO 2008087257). It is realized that the lectin according to the invention binds a specific limited glycan group. It is realized that the plant lectins ECA, UEA and DSA-1 share substantial homology as well as galectin-1 and galectins in general, and that numerous equivalent lectins of plant and/or animal or other eukaryotic origin, with substantial homology can be found. Preferred lectins includes lectins of Erythrina, Ulex, and Datura species or related plant species, or lectins of animal species, such as mammals or human, with similar specificity and at least 50%, 60%, 70%, 80%, or 90%, or 95% homology to ECA, UEA, DSA or galectin-1. Some preferred lectins related to Erythrina or Ulex plant family include lectins indicated in WO2010004096 and listed in Table 2 below. It is further realized that Datura family lectins share homology with the lectins, and equivalent lectins include also Datura lectin homologs within the desired range. The invention is further directed to a recombinant form and mutants of the lectins as defined in WO2010004096 and WO 2008087257, such as the mutant wherein N-glycosylation site is mutated to non-glycosylatable form lacking Asn-X-Ser/Thr glycosylation site, e.g. as defined in WO 2008087257, and WO2010004096, especially in FIG. 9.

It is noted that 3D structures of Erythrina family lectins are known (Shaanan et al. Science. 1991 254,862-6, PDB ID: 1LTE; PDB ID:3N36; Svensson et al J Mol Biol 2002,321, 69-83 PDB ID: 1GZC; PDB ID: 1UZZ). Blast search of mammalian proteins reveals homology of ECA lectin to galectins, e.g., galectin-8, such as human galactin-8, with protein access code EAW70057 having 44% coverage and 24% identity in blast search. In an embodiment, animal lectins or galectin homologous lectins homologous to ECA will be tested in the method of the invention, and thus selected and/or optimized for the methods of present invention. The galectins are homologous family of proteins and further examples includes human galectin-1 AAP36586.1 with reference to 3D structure in complex with glycan 2ZKN_A; rat galectin NP_{—}063969.1; Sus scrofa galectin-1 AAT37622.1; galectin-8 family proteins: human variant a NP_{—}006490.3, human b NP_{—}963837.1, Mus musculus NP_{—}061374.1; galectin-9: 2D6K_A, NP_{—}001152773.1, NP_{—}034838.2; galectin-3 3AYA_A, NP_{—}114020.1, NP_{—}001139425; galectin-4: NP_{—}034836.1, P56470.1, P38552.1; galectin-12 Q96DT0.1, Q91VD1.2; galectin-6 NP_{—}034837.1; galectin-2 NP_{—}079898.2; examples of other animal galectins frog galectin family lectins BAB83247.1, ACO51808.1, and other animals EHB02181.1, ACQ58967.1, or AEK50322.1. In an embodiment, the invention is directed to testing, selecting and/or optimization of other lectins with ECA like specificity for the methods of the invention, in an embodiment the lectin is a Ricinus communis agglutinin-1(RCA-1) type lectin such as AAB22584 with low sequence homology to ECA. In an embodiment, the lectins such as RCA-1 include lectin but lack toxin domain. It would also be of ordinary skill to analyze the polypeptide sequence, e.g. by Edman degradation or mass spectrometry, of commercial proteins such as Datura stramonium lectin 1 with regard to terminal Galβ4GlcNAc binding specificity.

In an embodiment, the preferred lectins of the invention do not bind or not effectively or not specifically bind, to the non-reducing end mannose, such as Mana or Manα2/3/6, or GlcNAc residues, or optionally further not to Galα4, GlcNAcα4, or optionally further not fucose conjugated to GlcNAc such as Lewis×Ga; β4(Fucα3)GlcNAc, or Lewis a Galβ3(Fucα4)GlcNAc, and optionally further not to Manα3(Manα6)Manβ4GlcNAcβ4GlcNAc, optionally further being modified to reducing end GlcNAc by Fucα6.

It is further realized that the protein domains (lectins, antibodies or enzymes, preferably of human or primate or mammalian origin) recognizing terminal glycan structure is quite rigid structure which defines the 3D structure of a lectin and other binding proteins. The binding structures preferably include polar residues such as Ser, Thr, Asp, Asn, Glu, Gln, Lys, Arg, His forming hydrogen bonds to hydroxyl and acetamido groups, in an embodiment the binding site such as lectin site further includes an aromatic amino acid residue such as phenylalanine or tyrosine forming a hydrophobic and/or glycan ring stacking interaction.

In an embodiment, the invention is directed to a complex of the glycan binding substance such as lectin, antibody or enzyme and differentiating or differentiated retina cells, preferably so that the binder/lectin is capable of binding terminal glycan epitopes of the cells and/or cell matrix, and optionally wherein the binding substance is bound to the cells and/or cell matrix. The terminal glycan epitopes are preferably conjugated to proteins and/or lipids of the cells. Optionally, the complex is comprised in a cell culture comprising in some embodiments at least one, two, three, four, or five cell culture media component or component class selected from a group consisting of growth factors, proteins, glycoproteins or glycoprotein fraction or component thereof, knock-out-serum, nutrients, amino acids sugars or any other cell culture media component described in the invention. In an embodiment, the invention is directed to cell culture comprising the binding substance and retina type cells, optionally characterized by any one of the markers as defined in examples or cell differentiating to retina cells, and optionally comprising cell culture media comprising nutrients such as essential nutrients including amino acids and/or sugar.

The amount of binder, such as lectin, used in a solution is about 0.1-500 μg/ml, preferably about 5-200 μg/ml or about 10-150 μg/ml. The amount of binder for immobilization of the cell culture surface is about 0.001-50 μg/cm², preferably from about 0.01-50 μg/cm² to about 0.1-30 μg/cm², more preferably about 0.3-10 μg/cm² for a lectin with Mw of about 50 kDa, or corresponding molar density per surface area used. In one embodiment, about 1-50 μg/cm², or about 5-40 μg/cm², preferably about 10-40 μg/cm² of binder is used in a solution to coat a plastic cell culture surface. In one embodiment, the concentration in the coating solution is between about 50-200 μg/ml for a binder with Mw of about 50 kDa or corresponding molar density per surface area used. In a specific embodiment, a plastic cell culture well with polystyre surface is coated by passive adsorbtion using about 140 μg/ml solution in amount of about 30 μg/cm² for a binder with Mw of about 50 kDa.

In an embodiment, the cells are cultivated for at least 5, 10, 20, or 30 days, and in other embodiments at least 35, 40, 45, 50, 55, or 60 days. In an embodiment, the cell culture temperature varies from 30-40, or 33-39, or 35-38 degrees of Celsius. In an embodiment, the amount of CO2 in atmosphere is between 2-8%, 3-7% or 4-6% or about 5%. In an embodiment, the cells are cultivated in form of a layer. In an embodiment, the cell culture media is serum free, and optionally contains a glycoprotein fractions such as a glycoprotein fraction, e.g., an isolated glycoprotein comprising fraction or a recombinant glycoprotein fraction. Accordingly, the cell culture media can comprise transferrin and/or albumin. In an embodiment, said glycoprotein fraction comprises animal type glycosylation and optionally major part of the animal type glycosylation does not bind to the binder substances of the invention, or to the lectins of the invention. In an embodiment, the glycoprotein fraction is or is comprised in a “knock-out serum” or “ko-SR” preparation.

The starting cellular material can be any type of human pluripotent stem cell, in one embodiment, it is embryonic stem cell or cell line derived thereof. To avoid any possible ethical problems related to establishing ESC lines it is currently possible to create them without destroying the embryo as described e.g. by Klimanskaya et al (Nature 444: 481-485; 2006). In a preferred embodiment, ESC lines are established without destroying the embryo. In another preferred embodiment of the invention the starting cell is an iPS cell.

The present invention further relates to a use of lectin ECA or binders with a binding specificity essentially similar to ECA, as an in vitro cell culture reagent or additive for production of RPE cells or their progenitors from pluripotent stem cells. The invention is further directed to any lectins and uses thereof with the binding specificity according to the invention. The invention is further directed to uses and compositions wherein the lectin has binding specificity essentially similar to specificity of Ertyhrina, Datura, or Ulex -plant or ECA, UEA or DSA-1 lectins or binding specificity of galectins, such as galectin-1.

The invention is especially directed to essentially similar binding towards the terminal epitopes according to Formula (Fucα2)ₙGalβ4GlcNAc, wherein n is 0 and/or 1 and similar non-binding character especially to the non-binding epitopes defined in the invention. In an embodiment the binding molecule or the lectin binds both of the non-reducing end terminal structures (n is 0 and n is 1), the substantial binding is preferably at least 1, 2, 3 or 4, or 5 orders of magnitude stronger than background/non-binding, when measured by a solid phase assay e.g. as used with glycan arrays by Consortium of Functional Glycomics or as defined in WO/2009/060129.

In a preferred embodiment of the invention, the invention is related to a cell culture vessel, whose surface, in particular those surfaces onto which cells can be assumed to be in contact, is covered with lectin ECA or binders with a binding specificity essentially similar to that of ECA. The pluripotent stem cells are cultivated in the RPE-inducing cell culture medium in the said cell culture vessel. It should be noted that there are a number of cell culture media suitable for RPE differentiation known in the art. It is of further note that specifications, such as the material or size of the cell culture vessel can vary. In one embodiment of the invention the vessel is a cell culture plate or flask made of plastic. In another embodiment, the vessel is a fermentor type of device. Furthermore, it is well known in the art that the lectin can be attached onto surface by various methods known in the art; the attachment can be passive or active, such as covalent linkage.

### EXAMPLES

### Example 1

### Lectin ECA Allows Adherent Differentiation of Retinal Pigment Epithelial Cells From Human Pluripotent Stem Cells

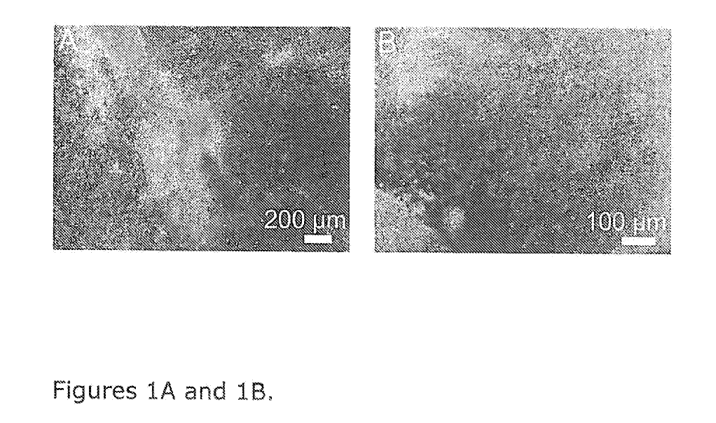

Undifferentiated Regea 08/023 hESCs were manually cut and plated onto ECA and Matrigel coated cell culture wells in retinal pigment epithelium differentiation medium (RPEbasic) identical to hESC culture medium (see below) with the modifications of 15% ko-SR and no bFGF (Vaajasaari et al., 2011, J Molecular Vision 17:558-575). The cells were cultured at 37° C., 5% CO₂ and the RPEbasic medium was changed three times a week. The day of the appearance of the first pigmented cells and the average number of pigmented areas per well on days 21, 28, 35 and 50 were observed (Table 1). Cells were pictured with Nikon Eclipse TE2000-S phase contrast microscope and NIS-Elements D 3.1 software (Nikon Instruments Europe B.V Surrey, England) on day 28 (FIG. 1A). The pigmented areas were manually cut off and selectively replated to new similarly coated wells on day 50 to create selected population of mature RPE cells on day D70 (FIG. 1B).

Basic hESC culture medium consists of Knockout Dulbecco's Modified Eagle Medium containing 20% KO-SR, 2 mM Glutamax, 0.1 mM 2-mercaptoethanol (all from Invitrogen, Carlsbad, Calif.), 1% Minimum Essential Medium nonessential amino acids, 50 U/ml penicillin/streptomycin (both from Cambrex Bio Science, Walkersville, Md.), and 8 ng/ml human basic fibroblast growth factor (bFGF; R&D Systems Inc., Minneapolis, Minn.).

### Example 2

### Neuroectodermal and Retinal Precursor Marker Expression Is Increased On ECA Cultures As Compared To Cells Cultures On Standard Matrigel

RNA samples were collected on days 7 and 28 of differentiation for quantitative PCR analysis of RPE differentiation related genes. Total RNA was extracted using NucleoSpin XS-kit (Macherey-Nagel, GmbH & Co, Duren, Germany) according to the manufacturer's instructions. All cells on a well were collected, lysed to lysis buffer and stored at −70° C. prior the RNA extraction. RNA from undifferentiated Regea 08/023 was collected to serve as calibrator sample from the population of cells initially plated to the matrices. The RNA concentration and quality were assessed with NanoDrop 1000 spectrophotometer (NanoDrop Technologies, Wilmington, Del., USA).

200 ng of RNA was translated to cDNA with High-Capacity cDNA Reverse Transcription Kit (Applied Biosystems) according to manufacturer's instructions. Taqman gene expression assays PAX6 (Hs00240871_m1), RAX (Hs00429459_m1) and MITF (Hs01115553_m1) were used for q-PCR reactions. GAPDH (Hs99999905_m1) was used as a housekeeping control (All from Applied Biosystems, Life Technologies, Carlsbad, Calif., USA). The PCR reaction consisted of 3 μl of cDNA in 1:5 dilution, 7.5 μl of 2× TaqMan Universal PCR Master Mix (Applied Biosystems), 0.75 μl of assay and 3.75 μl of H₂O. All samples and no template controls were analyzed as three replicates. The q-PCR was carried out with Applied Biosystems 7300 Real-time PCR system: 2 min at 50° C., 10 min at 95° C., and 40 cycles of 0.15 min at 95° C. and 1 min at 60° C.

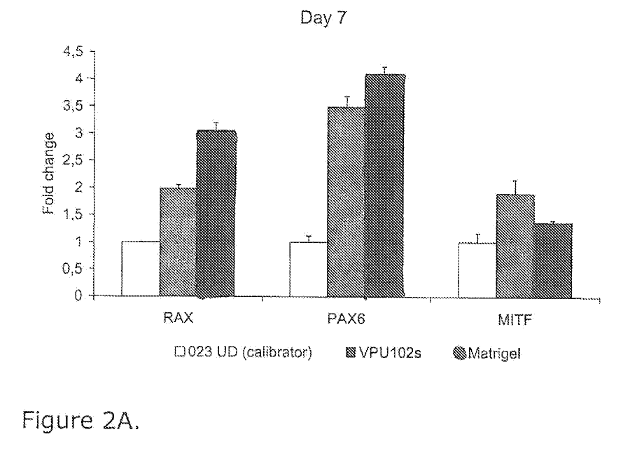

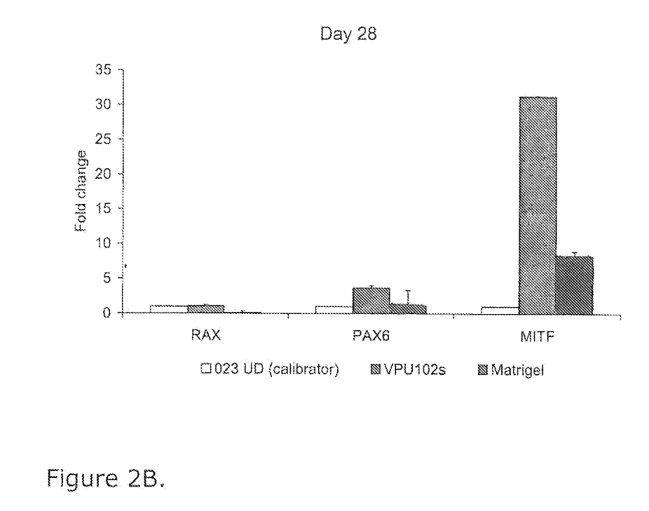

The data was analyzed with 7300 System SDS Software (Applied Biosystems) and Microsoft Office Excel 2003 (Microsoft Corporation). Relative quantification was calculated with the −2^{ΔΔCt} method (Livak and Schmittgen. Methods. 2001; 25:402-8). The data was normalized to the expression of GAPDH within a sample. The data is presented as fold change values comparing to expression level of undifferentiated hESCs set as 1. Standard deviation of the 3 replicate fold change values is presented as error bars. Neuroectodermal (PAX6) and retinal (RAX) precursor genes as well as early marker for RPE cells were upregulated on ECA on day 7 (FIG. 2A). On day 28, expected decreasing expression of RAX and PAX6 and enhanced expression of MITF was detected. Increased expression of MITF was higher in cells cultured on ECA as compared to matrigel (FIG. 2B).

### Example 3

### ECA Allows Adherent Differentiation and Maturation of RPE Having Typical Morphology Characteristics For These Cells

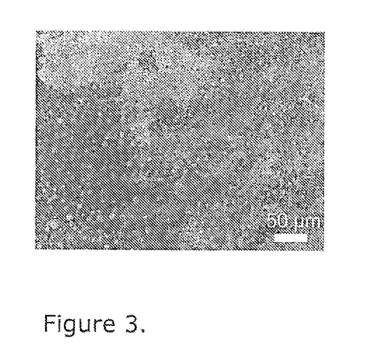

During onset of the differentiation, the pigmented areas were manually cut off and selectively replated to new similarly coated ECA wells on day 50 and again on day 91 to create a pure population of mature RPE cells demonstrating typical cobblestone morphology of RPE cells (FIG. 3).

### Example 4

### Immunostainings Demonstrating Maturated RPE Phenotype of Differentiated Cells

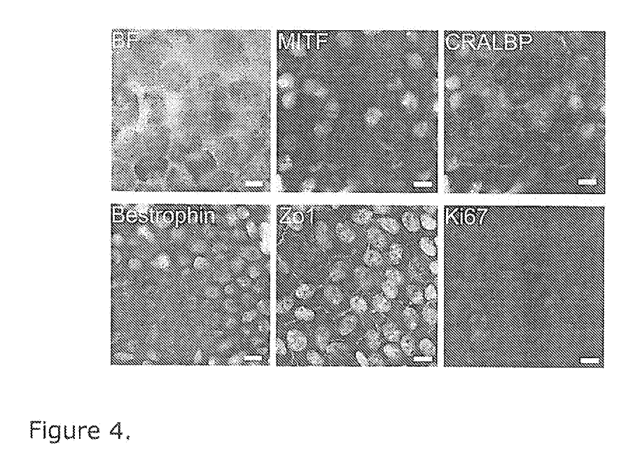

Human ESC-derived RPE cells after 140 days of differentiation on ECA were immunostained for expression of RPE-specific markers: MITF, CRALBP, Bestrophin, tight junction protein Zo1, and proliferation marker Ki67 (FIG. 4). The nuclei were counterstained with DAPI. Scale bars are 10 μm. The results indicated that the cells cultivated had phenotype typical to RPE cells.

TABLE 1
Pigmentation rate of hESCs differentiating on ECA and Matrigel.

| | First | | | | |
|---|---|---|---|---|---|
| | pigmentation | | | | |
| | observed on | Average number of pigmented areas/well | | | |
| | day | Day 21 | Day 28 | Day 35 | Day 50 |
| ECA | 15 | 0.7 | 2.2 | 3.8 | 10.3 |
| | | (n = 6) | (n = 6) | (n = 4) | (n = 4) |
| Matrigel | 16 | 0.2 | 1 | 3.8 | 13.5 |
| | | (n = 5) | (n = 5) | (n = 4) | (n = 4) |

N = number of wells

Table 2 shows a list of lectins whose amino acid sequences are highly homologous to that of ECA (see WO2010004096).

ECA|1UZY|A
ECO|1AX0|A
Erythrina_variegata lectin
WBAI|O24313.1|LEC1_PSOTE
WBAII|1FAY|A
Phaseluna|CAA93830.1
PhaseAugusti|CAH59200.1
Phasemacu|CAH60256.1
PhaseLepto|CAH60214.1
PhaseVulg|CAD28674.1
Soy|2SBA|A
Robinia|BAA36415.1
Maackia|AAB39934.1
UlexII|AAG16779.1
UlexGene1Ulex
UlexI|1FX5|A
SOPJA|P93535.1|LECS_SOPJA

1. A method of producing human retinal pigmented epithelial cells in vitro wherein pluripotent human stem cells are cultured in a medium initiating the differentiation of the cells into retinal pigmented epithelial cells and in the presence of a binder binding to terminal N-acetyllactosamine Galβ1-4GlcNAc and/or blood group H determinant type 2 Fucα1-2Galβ1-4GlcNAc.

2. The method according to claim 1, wherein said binder is lectin.

3. The method according to claim 2, wherein said lectin is or has binding specificity of ECA, UEA, DSA-1 or galectin-1.

4. The method according to claim 1, wherein said pluripotent human stem cells are human embryonal stem cells.

5. The method according to claim 1, wherein said pluripotent human stem cells are induced pluripotent stem (iPS) cells.

6. The method according to claim 1, wherein said medium initiating the differentiation of the cells into retinal pigmented epithelial cells is a modified hESC culture medium with 15% ko-SR and no bFGF.

7. Use of a binder binding to terminal N-acetyllactosamine (Galβ1-4GlcNAc) and/or blood group H determinant type 2 (Fucα1-2Galβ1-4GlcNAc) as an in vitro cell culture reagent or additive for production of human retinal pigmented epithelial cells or their progenitors from pluripotent stem cells.

8. The use according to claim 7, wherein said binder is lectin.

9. The use according to claim 8, wherein said lectin is or has binding specificity of ECA, UEA, DSA-1 or galectin-1.

10. The use according to claim 7, wherein said pluripotent human stem cells are human embryonal stem cells or induced pluripotent stem (iPS) cells.

11. A cell culture system for producing retinal pigmented epithelial cells in vitro wherein it comprises:
a) cell culture vessel coated with a binder or binders binding to terminal N-acetyllactosamine (Galβ1-4GlcNAc) and/or blood group H determinant type 2 (Fucα1-2Galβ1-4GlcNAc) and in said culture vessel:
b) a human pluripotent stem cell; and
c) medium suitable for initiating the differentiation of human pluripotent stem cells into retinal pigmented epithelial cells.

12. The cell culture system according to claim 11, wherein said binder is lectin.

13. The cell culture system according to claim 12, wherein said lectin is or has binding specificity of ECA, UEA, DSA-1 or galectin-1.

14. The cell culture system according to claim 11, wherein said pluripotent human stem cells are human embryonal stem cells or induced pluripotent stem (iPS) cells.

15. A composition wherein said composition comprises a complex of a binder capable of binding to terminal N-acetyllactosamine Galβ1-4GlcNAc and/or blood group H determinant type 2 Fucα1-2Galβ1-4GlcNAc and differentiated human retina cells, wherein said binder is bound on the surface glycans of said retina cells.

16. The composition according to claim 15, wherein said binder is lectin.

17. The composition according to claim 16, wherein said lectin is or has binding specificity of ECA, UEA, DSA-1 or galectin-1.
